(19) United States
(12) Patent Application Publication
(10) Pub. No.: US 2005/0272142 A1
(43) Pub. Date: Dec. 8, 2005

(54) Fluid vessel

(75) Inventor: Takashi Horita, Osaka (JP)

Correspondence Address: BIRCH STEWART KOLASCH & BIRCH, PO BOX 747, FALLS CHURCH, VA, 22040-0747, US

(73) Assignee: HITACHI MAXWELL, LTD., Osaka (JP)

(21) Appl. No.: 11/104,448

(22) Filed: Apr. 13, 2005

(30) Foreign Application Priority Data
Apr. 14, 2004 (JP) P2004-118724
Apr. 14, 2004 (JP) P2004-118725
Jun. 18, 2004 (JP) P2004-180467

(51) Int. Cl. G01N 15/06; C12M 1/34

(52) U.S. Cl. 435/287.1; 422/68.1

(57) ABSTRACT

A fluid vessel includes a base member in the form of a plate of which one surface has a concave channel and a cover plate which is bonded to the base plate so as to close an opening surface of the channel. The base member and the cover plate are bonded through a double-faced adhesive sheet. The double-faced adhesive sheet has an opening which corresponds to a formation pattern of the channel. The double-faced adhesive sheet includes a flat and smooth sheet substrate which is made of a plastic sheet having a chemical resistance as well as adhesive layers which are laminated onto both surfaces of the sheet substrate. In a state in which the cover plate is bonded to the base member, a space between the cover plate and a periphery of the channel opening is filled with an inner peripheral wall portion which defines the opening of the double-faced adhesive sheet.

### CROSS-REFERENCE TO RELATED APPLICATIONS

The present application claims priorities under the Paris Convention based on Japanese Patent Application No. 2004-118724 (filing date: Apr. 14, 2004, title: fluid device) Japanese Patent Application No. 2004-118725 (filing date: Apr. 14, 2004, title: fluid device) and Japanese Patent Application No. 2004-180467 (filing date: Jun. 18, 2004, title: production method of fluid device), and the contents of those applications are incorporated herein by reference in their entirety.

### BACK GROUND OF THE INVENTION

1. Field of the Invention

The present invention relates to a fluid vessel which is used to carry out for example a medical analysis wherein an analyte is subjected to absorption and purification for the DNA analysis, a reaction of a chemical, and an analysis of a synthesized chemical. When a reaction of a fluid is carried out in such fluid vessel, the fluid vessel according to the present invention may be referred to as a reaction vessel. Such kind of reaction vessels may be referred to also as a biochip, a microchip, or the like. It is noted that the “fluid” in the present specification includes a liquid, and a fluid which may be handled as a liquid (such as a liquid which contains fine solids, a liquid which contains fine liquid droplets and/or fine gas bubbles as the other phase, a solution and the like). Also, the fluid in the specification may be a gas (which may include fine solids, fine liquid droplets or the like).

In the description of the present specification as well as the claims which will be recited below, “liquid” is used as a term which generically means a liquid and a fluid which may be handled as a liquid as described above.

2. Description of the Related Art

Recently, approaches to carry out more effective medical treatments have been rapidly developed based on results of a structure analysis of DNA, and a number of compact reaction vessels have been developed for the structure analysis of DNA. Such reaction vessel generally has a structure in which a base member is bonded to a cover plate together, wherein the base member is in the form of a plate having a small channel (i.e. a micro-channel) in its surface, and the cover plate has an external profile which is nearly the same as that of the base member and closes an opening of the channel. The cover plate has a through hole which communicates with the channel so that a liquid such as an analyte, a reaction liquid or the like may be supplied into or discharged from the channel via the through hole, and the reaction or the analysis or the like is carried out while the liquid is contained in the channel. Such reaction vessel having the above mentioned structure is disclosed for example in following Patent References 1 to 5:
Patent Reference 1: Japanese Patent Kokai Publication No. 2002-159285 (see paragraph 0017, and FIG. 1);
Patent Reference 2: Japanese Patent Kokai Publication No. 2002-204945 (see paragraph 0041, and FIG. 1);
Patent Reference 3: Japanese Patent Kokai Publication No. 2003-279537 (see paragraph 0018, and FIG. 1);
Patent Reference 4: Japanese Patent Kokai Publication No. 2001-281233 (see paragraph 0027, and FIG. 1); and
Patent Reference 5: Japanese Patent Kohyo Publication No. 9-502795 (see the last paragraph of page 4, and FIG. 2).

Patent Reference 1 discloses a reaction vessel having a structure in which a base member in the form of a plate having a small channel formed in its surface is bonded to a cover plate so that an opening of the channel is closed. For such bonding, an adhesive is usually used (see for example, Patent Reference 3).

When the cover plate is bonded to the base member, the reaction vessel can be produced readily and less expensively, and better sealing of a space between joint surfaces of the base member and the cover plate is ensured because the adhesive occupies the space widely. Especially, when an adhesive which contains an ultraviolet (UV) curable resin is used, any arbitrarily appropriate period from its application to its curing can be selected, which is also preferable in the workability and also the processability.

When the cover plate is bonded to the base member, it is difficult to apply the adhesive uniformly with a selected thickness. In the case wherein an adhesive of which viscosity is small is used, the capillary phenomenon which happens when the cover plate is brought close to the base member makes the uniform application of the adhesive possible, but the adhesive is likely to penetrate into the channel which is important.

An adhesive having a high viscosity may be applied in a predetermined pattern when using a small amount ejecting device and an industrial robot. What is problem is that the adhesive which has been already applied cannot be spread sufficiently between the cover plate and the base member when the cover plate is placed on the base member to join them, so that an unwanted space may be formed around a periphery of an opening of the channel. To the contrary, when a more amount of the adhesive is applied, the spread adhesive may penetrate into the channel from the periphery of the opening of the channel.

As a manner with which the base member is integrated with the cover plate, other bonding manner except the above manners, and a heat welding manner may be employed. For example, when the base member is formed of a plastic material which is compatible to a plastic material of the cover plate, a solvent is applied to any one of the base member and the cover plate so as to dissolve a surface to be bonded and then they are brought together to be bonded. Alternatively, the base member and the cover plate are ultrasonically welded while they are in contact with each other, so as to integrate them together.

However, the bonding manner with using the solvent may produce a space in the closed channel is contaminated with residual of the solvent, and the residual has to be removed by an additional operation such as washing. Further, there is a possibility that a peripheral portion around the opening of the channel which portion is in the dissolved state may be pushed into the channel when the cover plate is pressed against the base member. In addition, when the solvent is a toxic one, a safety matter cannot be negligible.

When the base member and the cover plate are brought integral by means of the ultrasonic welding, there is a possibility that dusts (or melt droplets) formed by the ultrasound are confined in the space of the channel, so that a contamination problem of the space in the channel arises. Further, any of the above bonding manners cannot be applied when one of the base member and the cover plate is made of a glass material, a ceramic material, a metal material or the like.

In addition, when the flatness of the base member or the cover plate is not so good, there occurs a gap between the base member and the cover plate so that sealing between them may be insufficient. Such insufficiency may be solved by improving the flatness of the base member, which increases the processing cost of the base member so that the production cost of the reactor increases. In the bonding manner of using the adhesive, it is noted that, depending on the adhesive to be used, the base member and the cover plate have to be fixed with using a tool so as to keep their positional relationship until the adhesive cures, which requires an additional operation so that the production cost increases.

### DISCLOSURE OF THE INVENTION

### SUMMARY OF THE INVENTION

It is an object of the present invention to provide a fluid vessel, and particularly a reaction vessel, as to which at least one of the following is improved: prevention of the penetration of the adhesive into the channel upon bonding the base member and the cover plate; prevention of the formation of the unwanted space around the periphery of the opening of the channel; prevention of the dissolution of the adhesive into an agent in the channel; and prevention of the reaction of the adhesive with an analyte in the channel.

It is another object of the present invention to provide a fluid vessel, and particularly a reaction vessel wherein the cover plate is appropriately bonded to the base member, and particularly even when the number of the fluid vessels to be produced is small, they are produced less expensively.

It is a further object of the present invention to provide a fluid vessel, and particularly a reaction vessel wherein an adhesive layer is more appropriately formed with a more uniform thickness. It is other object of the present invention to provide a fluid vessel, and particularly a reaction vessel wherein the cover plate is bonded to the base member while being more appropriately located on the base member, so that occurrence of failure upon locating the cover plate on the base member is suppressed whereby the production yield of the vessels is improved, and also so that the vessels are produced without a problem in integrating the base member and the cover plate even when they are made of different materials.

It is a further object of the present invention to provide a fluid vessel, and particularly a reaction vessel which is readily produced with a less trouble compared with the production of the same kind of vessels in the conventional process, which leads to the less production cost of the vessels.

The present invention provides a fluid vessel, and particularly a reaction vessel which is improved as to at least one of the above described various objects, and also provides a production method of such fluid vessel, and particularly such reaction vessel.

The fluid vessel, and particularly the reaction vessel according to the present invention comprises:
a base member having a channel on its one surface which channel is able to receive for example an analyte;
a cover plate which closes an opening surface of the channel and which is located above the base member; and
a bonding element which joins the base member and the cover plate. The channel is able to receive a liquid such as the analyte and formed by providing the base member with a concave portion.

In the first aspect, the fluid vessel according to the present invention comprises, as the bonding element, a double-faced adhesive sheet which is placed between a joint surface of the base member and a joint surface of the cover plate. See reference number “6” in FIGS. 1 to 3 which will be referred to later. The double-faced adhesive sheet has an opening which has been formed beforehand so that a profile of such opening corresponds to a profile of the opening surface of the channel of the base member. It is noted that the opening surface of the channel corresponds to an exposed surface of a space defined by the channel. The fluid vessel in which the cover plate is joined with the base member is characterized in that a space between the cover plate and a periphery portion of the channel which portion defines the opening surface of the channel is occupied by an inner peripheral wall portion of the double-faced adhesive sheet which portion defines the opening of the double-face adhesive sheet, so that such space is filled with the double-faced adhesive sheet. It is noted the double-faced adhesive sheet is a bonding member comprising a sheet substrate which has a layer of an adhesive (usually, a pressure sensitive adhesive) on its each side, and it is in a thin sheet form as a whole.

In a preferable embodiment of the first aspect according to the present invention, the double-faced adhesive sheet is composed of the sheet substrate and the adhesive layers which are laminated integrally onto the both sides of the substrate. In this embodiment, it is preferable that the sheet substrate is made of a plastic sheet which does not easily bend and also which is excellent in its chemical resistance.

In another preferable embodiment of the first aspect according to the present invention, the inner peripheral wall portion of the double-faced adhesive sheet which portion defines the opening of the adhesive sheet is coated with a resin layer which is of a chemical resistance.

It is noted that the above preferable features are used alone respectively, or in any of various combinations thereof as far as such combination is possible.

In the second aspect, the fluid vessel of the first aspect according to the present invention comprises, in place of the double-faced adhesive sheet, an adhesive layer which is formed on at least one of the joint surface of the base member and the joint surface of the cover plate, and preferably on the former joint surface, and such adhesive layer is formed by the screen printing manner which is applied to the joint surface except the opening of the channel.

In a preferable embodiment of the second aspect according to the present invention, the cover plate is made of a transparent material, and the adhesive layer is made of an ultraviolet (UV) curable adhesive. As such adhesive, an epoxy based UV curable adhesive which is of a chemical resistance is preferably used.

It is noted that the above preferable features are used alone respectively, or in any of various combinations thereof as far as such combination is possible.

In the third aspect, the fluid vessel of the second aspect according to the present invention comprises the base member and the cover plate each of which is made of a thermoplastic material. For example, each of the base member and the cover plate is a molded product of such a resin. Similarly, the bonding element is an adhesive layer which is formed on at least one of the joint surface of the base member and the joint surface of the cover plate, and the base member and the cover plate are bonded together through at least two spot-welded portions (see FIGS. 7 and 8 which will be referred to later, and especially spot-welded portions indicated with reference number 22).

In a preferable embodiment of the third aspect according to the present invention, one of the joint surfaces of the base member and the cover plate has the spot-welded portion which is formed by spot-welding a small convex portion which abuts against the other joint surface. The other joint surface may have a small concave portion where the small convex portion abuts. In a particularly preferable embodiment, the adhesive layer and the spot-welded portions derived from the small convex portions are formed on one surface of the base member, and such adhesive layer is present on at least a periphery of the channel except the spot-welded portions.

In a preferable embodiment of the third aspect according to the present invention, the cover plate is made of a transparent thermoplastic resin material, and the adhesive layer is made of an ultraviolet curable adhesive, wherein the base member and the cover plate are bonded by means of spot-welding first, and then the adhesive layer is cured by the irradiation of UV.

In a other preferable embodiment of the third aspect according to the present invention, spot-welding is carried out by ultrasonic welding.

It is noted that the above preferable features are used alone respectively, or in any of various combinations thereof as far as such combination is possible.

As to the fluid vessel according to any of the above aspects of the present invention, in a preferable embodiment, the base member is in the form of a rectangular or square plate, and the cover plate has a projected planar shape which is the same as that of the base member. In other preferable embodiment, the joint surfaces of the base member and the cover plate are made flat and smooth. It is noted that these preferable features are used alone respectively, or in any of various combinations thereof as far as such combination is possible.

As obviously understood by those skilled in the art, the fluid vessel according to the present invention has a port through which the fluid is supplied into the space formed by closing the channel of the base member with the cover plate. In addition, it is preferable that the fluid vessel has a port through which thus supplied fluid is discharged or through which a gas (usually air) having been present in the space is discharged when a fluid is supplied into the space, that is a so-called airing port. Such supply port and such optional discharge port are provided to any one of the base member and the cover plate, or to the both of them. Usually, the port(s) are provided by forming a hole(s) through the base member and/or the cover plate which hole(s) opens to the channel.

The present invention further provides a production method for producing the fluid vessel of any one of the above mentioned first to third aspects according to the present invention.

The fluid vessel of the first aspect is produced by a method for producing a fluid vessel comprising:
preparing the above mentioned base member and also the above mentioned cover plate;
providing the double-faced adhesive sheet onto at least one of and usually either one of the joint surface of the base member and the joint surface of the cover plate except the region thereof which is to face to the channel;
superimposing the base member and the cover plate while they are aligned so that the double-faced adhesive sheet is located between the joint surface of the base member and the joint surface of the cover plate; and
optionally, pressing the base member and the cover plate against to each other.

The fluid vessel of the second aspect is produced by a method for producing a fluid vessel comprising:
preparing the above mentioned base member and also the above mentioned cover plate;
forming the adhesive layer, in the screen printing manner, onto at least one of and usually either one of the joint surface of the base member and the joint surface of the cover plate except the region thereof which is to face to the channel;
superimposing the base member and the cover plate while they are aligned so that the adhesive is located between the joint surface of the base member and the joint surface of the cover plate; and
optionally, curing the adhesive.

The fluid vessel of the third aspect is produced by a method for producing a fluid vessel comprising:
preparing the above mentioned base member and also the above mentioned cover plate each of which is made of the thermoplastic material;
providing the adhesive layer, in the screen printing manner, onto at least one of and usually either one of the joint surface of the base member and the joint surface of the cover plate except the region thereof which is to face to the channel;
superimposing the base member and the cover plate while they are aligned so that the adhesive is located between the joint surface of the base member and the joint surface of the cover plate;
partly connecting the joint surfaces of the base member and the cover plate by spot-welding at at least two points between the joint surfaces; and
further bonding the joint surfaces by means of the adhesive layer.

It is noted that the provision of the adhesive layer may be carried out in any appropriate manner. For example, the screen printing manner, the pad printing manner, the offset printing manner and the like may be used. Among these manners, the screen printing manner is preferable in that a thicker adhesive layer may be formed.

It is noted that the explanations mentioned above and below as to the fluid vessel according to the present invention are similarly applicable to any of the above mentioned production methods as far as they are applicable.

Effect of the Invention

As to the fluid vessel of the first acpect according to the present invention, the double-faced adhesive sheet as the bonding element bonds the cover plate to the base member so as to join them. In addition, since the double-faced adhesive sheet has the opening which has been formed beforehand so as to correspond to the profile of the opening surface of the channel formed in the base member, the space between the peripheral portion of the opening of the channel and the cover plate is occupied by the inner peripheral wall portion which defines the opening of the double-faced adhesive sheet in the fluid vessel in which the cover plate has been bonded to the base plate.

When the cover plate is bonded to the base member by means of the double-faced adhesive layer as the bonding element as described above, the following may be prevented:
the penetration of the adhesive into the channel is prevented upon bonding the cover plate to the base member,
the dissolution of the adhesive into the fluid (such as an agent) supplied into the channel of the fluid vessel; and/or
the reaction of the adhesive with such fluid. Further, by the correspondence of the profile of the opening surface of the channel to the opening profile of the double-faced adhesive sheet, the problem in the formation of the unwanted space around the opening surface of the channel may be eliminated.

Thus, in the fluid vessel of the firs aspect according to the present invention, the cover plate and the base member are bonded together, and the channel has a predetermined volume and the adverse effect of the bonding element on the fluid (such as an analyte) can be minimized, so that the fluid vessel (for example, the reaction vessel) having a good reliability is provided. Since the cover plate is able to bonded to the base member appropriately by means of the double-faced adhesive sheet, the integration of the base member and the cover plate can be carried out conveniently with less trouble, so that the fluid vessel may be produced less expensively even though the number of the fluid vessel produced in one lot is small.

Generally, a plastic sheet is less bendable and more elastic when compared with paper, a non-woven fabric or the like. Thus, when a flat and smooth plastic sheet which is excellent in its chemical resistance is used as the sheet substrate for the double-faced adhesive sheet, handling of the double-faced adhesive sheet is more readily upon the bonding operation compared with a case wherein the sheet substrate made of paper, a non-woven fabric or the like is used for the double-faced adhesive sheet, so that bonding can be carried out more rapidly while the double-faced adhesive sheet being located more accurately. Therefore, the penetration of the adhesive layer of the double-faced adhesive sheet into the channel can be surely prevented, and no unwanted space is formed around the opening surface of the channel. Further, even when the material for the base member is different from that for the cover plate, they are bonded together without a problem.

When an exposed surface of the inner peripheral wall portion which defines the opening of the double-faced adhesive sheet is coated with the resin layer which has the chemical resistance, the dissolution of the adhesive into the fluid (such as an analyte) from the adhesive layer of the double-faced adhesive sheet, and/or the unintended reaction of the adhesive with the fluid is surely prevented. Thus, using the fluid vessel according to the present invention leads to stable analytical experiments. Such resin coating may be formed by for example an immersion treatment.

In the fluid vessel of the second aspect according to the present invention, the adhesive layer which bonds the base member to the cover plate is formed by the screen printing manner, a thinner adhesive layer having a uniform thickness can be formed in an accurate pattern. The adhesive layer has to be formed on the joint surface of the base member and the cover plate except the region of the opening surface of the channel in either one of forming the adhesive layer on the base member and forming the adhesive layer on the cover plate, and the penetration of a portion of the adhesive layer into the channel can be suppressed when the cover plate is bonded to the base member. As a result, the dissolution of the adhesive into the fluid and/or the reaction of the adhesive with the fluid can be prevented as much as possible. Further, no unwanted space is formed around the opening surface of the channel.

Thus, the channel has a predetermined volume and the adverse effect of the adhesive on the fluid is minimized, so that the fluid vessel (for example, the reaction vessel) having a good reliability is provided. With the application of the adhesive in the screen printing manner, the base member and the cover plate are readily integrated, which reduces the production cost of the fluid vessel. Further, even when the material for the base member is different from that for the cover plate, they are bonded together without a problem.

In the fluid vessel of the second aspect according to the present invention, when the adhesive layer is formed of an ultraviolet curable adhesive, the adhesive layer does not cure without irradiation of UV. Therefore, the base member and the cover plate are accurately located, and then bonding is carried out to fix them, which improves the production yield of the fluid vessel. Since curing proceeds by the irradiation of UV, the contamination of the channel space by a residual organic solvent can be avoided. Such contamination cannot be avoided when bonding is carried out by an adhesive which contains an organic solvent. As a result, so that the fluid vessel having a high quality is provided without solvent contamination. Further, a safety related problem when using a toxic solvent is used can be also avoided. This is also applicable when the adhesive layer is made of a UV curable adhesive in the fluid vessel of the third aspect according to the present invention.

Further, in the case wherein the adhesive layer is made of the UV curable adhesive, when the cover plate is made of a transparent material, uniformly irradiating the whole of the adhesive layer with UV is possible so as to cure the adhesive layer effectively. In addition, a curing extent of the adhesive may be checked through the cover plate, or a reaction state of an analyte in the channel can be seen through the cover plate when a reaction is carried out with the fluid vessel, and therefore insufficient curing of the adhesive is prevented and also stable analysis becomes possible. In place of or in addition to the cover plate, the base member may be made of a transparent material.

When an epoxy based UV curable adhesive having a chemical resistance is used as the above mentioned UV curable adhesive, the reaction is suppressed even though the fluid in the channel contacts with the adhesive layer around the opening surface of the channel, so that for example the stable analysis becomes possible.

In the fluid vessel of the second aspect according to the present invention, when the adhesive layer is formed on the joint surface of the base member beforehand, no relative positional deviation between the channel and the adhesive layer has to be considered when the fluid vessel is produced, and bonding can be carried out with generally locating the cover plate on the base member, so that such bonding can be readily carried out. It is noted that when the adhesive layer is formed on the joint surface of the cover plate, the base member and the cover plate are bonded while they are carefully located such that the printed pattern of the adhesive layer aligns with the opening surface of the channel, which means that more labor is required for bonding.

Also, when the double-faced adhesive sheet is bonded to the base member in the production of the fluid vessel of the first aspect, or when the adhesive layer is formed on the base member in the production of the fluid vessel of the third aspect, it is convenient similarly to the above that the double-faced adhesive sheet is bonded to the joint surface of the base member beforehand, or the adhesive layer is formed on the joint surface of the base member beforehand, respectively.

In the fluid vessel of the third aspect according to the present invention, since the base member and the cover plate are not only bonded by the adhesive layer, but also linked by the spot-welding, they are integrated more securely. Thus, for example even when a large dropping impact force is applied to the fluid vessel, the base member and the cover plate are not separated. By spot-welding, the base member and the cover plate are firmly fixed even though the flatness of the joint surfaces is somewhat bad. With curing of the adhesive layer, the space between the joint surface of the base member and the joint surface of the cover plate is sufficiently filled, whereby good sealing between the joint surfaces is ensured.

In the fluid vessel of the third aspect according to the present invention, when the small convex portion is formed at a position on one of the joint surfaces at which position the spot-welding is to be carried out, efficient spot-welding of the base member with the cover plate becomes possible. When the cover plate is joined to the base member, the top of the small convex portion contacts with and abuts the other joint surface before the adhesive layer contacts the other joint surface. Thus, even when the cover plate is placed on the base member, the cover plate may be displaced so as to be surely located on the base member for bonding them. In addition, only the small convex portion is melted upon carrying out the spot-welding, reduction of the thickness or deformation of the cover plate or the base member may be prevented at the spot-welded portion.

In the production of the fluid vessel of the third aspect according to the present invention, when the base member and the cover plate are partly linked with the spot-welding before the bonding them by means of the adhesive layer, they do not have to be kept fixed by means of a tool during the curing operation of the adhesive layer, which reduces the production cost of the fluid vessel. That is, correction of the flatness of the joint surfaces of the base member and the cover plate, and fixing them for the purpose of alignment of such surfaces with using a tool, which are required upon the conventional bonding using only an adhesive layer, can be omitted, which reduces the production cost of the fluid vessel. Further, many kinds of tools are not required, which also reduces the production cost of the fluid vessel.

In the production of the fluid vessel of the third aspect according to the present invention, when the adhesive layer is formed on one joint surface except the spot-welded portion(s), the top of the small convex portion is allowed to surely contact with the other joint surface, so that the spot-welding can be carried out surely and readily without being disturbed by the adhesive layer. In addition, even when dusts (melt droplets) are formed by ultrasonic-welding which will be described below, it is blocked by the adhesive layer, so that no dusts penetrate into the channel. When the adhesive layer is formed around only on a peripheral portion of the opening surface of the channel, an amount of the adhesive to be used can be reduced so as to lower the production cost of the fluid vessel, and also difficulties in the formation of the adhesive layer can be minimized.

In the production of the fluid vessel of the third aspect according to the present invention, when the cover plate and the base member are the spot-welded in the ultrasonic-welding manner, the spot-welding can be carried out efficiently, which improves the mass-productivity of the fluid vessels.

In the fluid vessel of any aspect according to the present invention, when the cover plate is formed to have the same projected planar shape as that of the base member, the base member and the cover plate are appropriately located to each other by aligning outer profiles of their projected planar shapes upon bonding them.

In the fluid vessel of any aspect according to the present invention, when the joint surface of the base member (except the spot-welded portion(s) when it is present) and the cover plate (except the spot-welded portion(s) when it is present) are made flat and smooth respectively, the base member and the cover plate can be more firmly bonded while improving the adhesion by means of the adhesive layer or the double-faced adhesive sheet, so that the space the peripheral portion of the opening surface of the channel and the cover plate can be blocked tightly by the inner peripheral wall portion of the adhesive layer or the double-faced adhesive layer.

### BRIEF DESCRIPTION OF THE DRAWINGS

FIG. 1 shows an exploded perspective view of a fluid vessel of Example 1.

FIG. 2 shows a plan and partly cut-away view of the fluid vessel of Example 1.

FIG. 3 shows a cross-sectional view along a line A-A in FIG. 2.

FIG. 4 shows an exploded perspective view of a fluid vessel of Example 2.

FIG. 5 shows a cross-sectional view of the fluid vessel of Example 2

FIG. 6 shows a perspective view of a stencil plate used in Example 2.

FIG. 7 shows an exploded perspective view of a fluid vessel of Example 3.

FIG. 8 shows a plan and partly cut-away view of the fluid vessel of Example 3.

FIG. 9 shows a cross-sectional view along a line B-B in FIG. 8.

FIG. 10 shows a cross-sectional view which explains a production method of the fluid vessel of Example 3.

FIG. 11 shows an exploded perspective view of a fluid vessel of Example 4.

In the drawings, reference numbers indicate the following elements:
1 base member
2 cover plate
3 channel
4 through hole
6 double-faced adhesive sheet
7 sheet substrate
8 adhesive layer
9 opening
12 adhesive layer
13 relief margin
15 communication channel
22 spot-welded portion
23 small convex portion

### DETAILED DESCRIPTION OF THE INVENTION

### EXAMPLE 1

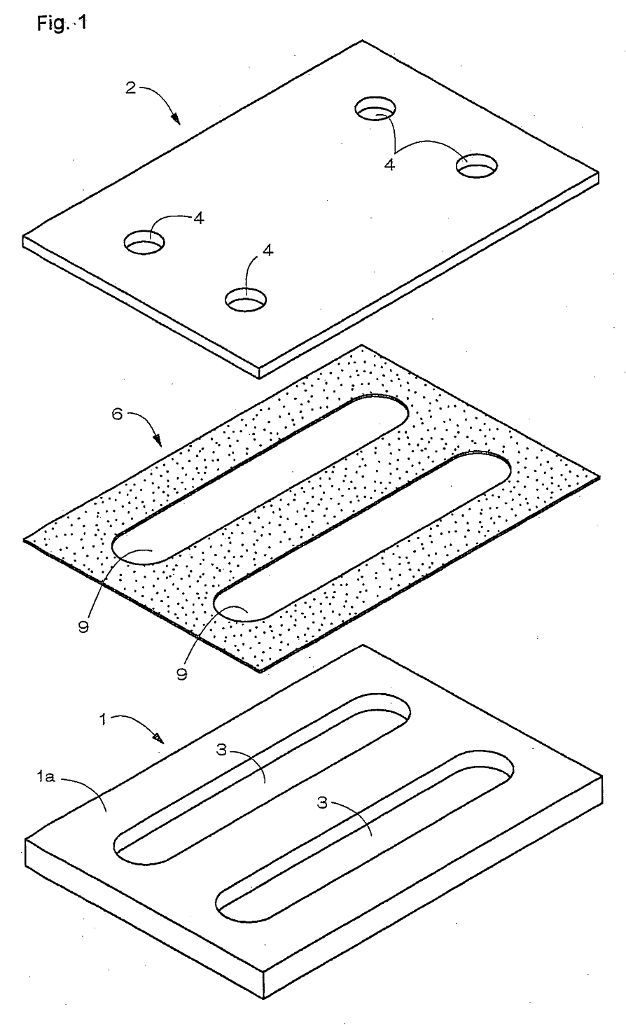

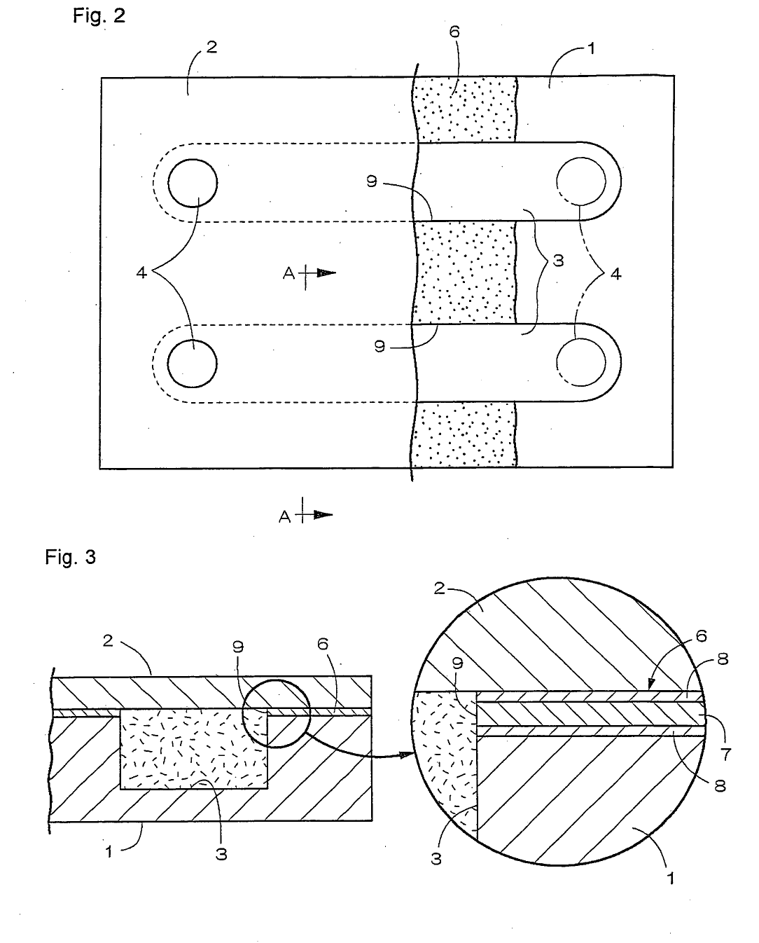

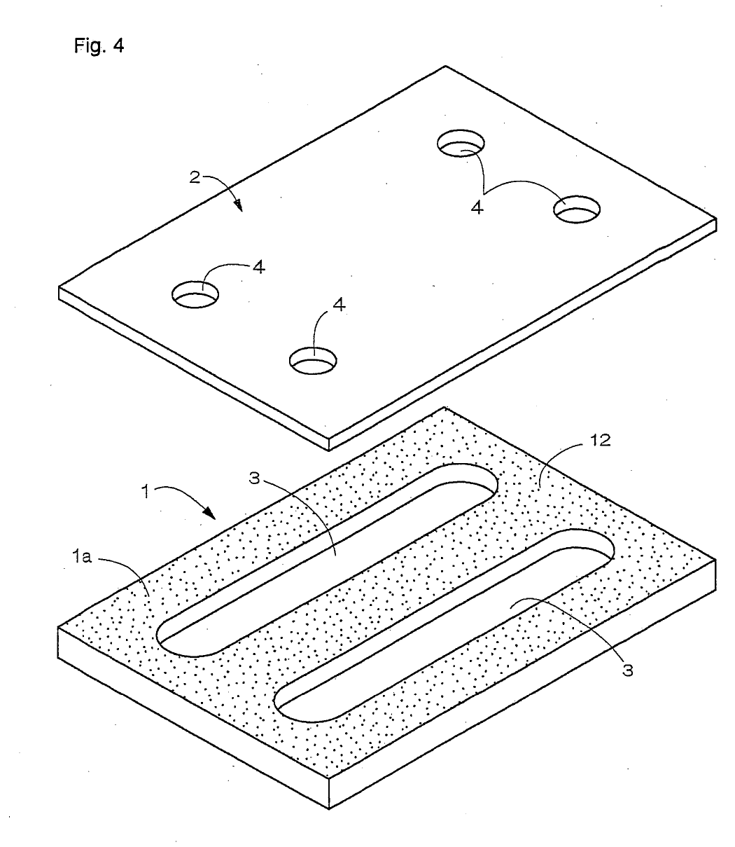

With reference to FIGS. 1 to 3, Example 1 of the fluid vessel (for example, a reaction vessel) according to the present invention which corresponds to the first aspect will be explained. It is noted that FIG. 1 schematically shows a perspective view of the fluid vessel in its exploded state, FIG. 2 schematically shows a plan view of the fluid vessel while a part thereof is cut away, and FIG. 3 schematically shows a cross-sectional view of a portion of the fluid vessel along a line A-A in FIG. 2. The fluid vessel of Example 1 comprises a base member 1 and a cover plate 2 which is bonded to the base member 1.

The base member 1 is formed into a plate form which defines a planar area and of which thickness dimension is relatively smaller than the other dimensions. The base member 1 has an elongated rectangular shape in its projected planar view. The base member 1 may be formed by using for example a borosilicate glass, a quartz glass or a resin such as a polydimetylsiloxane.

In FIG. 1, one surface 1a of the base member 1 which surface corresponds to the shown upper surface has been prepared to define a flat and also smooth area, and a plurality of channels 3 are formed as concave portions on said one surface 1a so that they can receive an amount of a fluid (for example, an analyte). Concretely, two elongated channels 3 and 3 are separately provided on the near side and the far side of said one surface 1a of the base member 1, respectively.

The cover plate 2 has the same planar shape as that of the base member 1, and for example, it is made of the same material as that of the base member 1. It is noted that the thickness dimension of the cover plate 2 is usually smaller than that of the base member 1 so that is of a thinner plate form.

The cover plate 2 is joined to said one surface 1a of the base member 1 so as to close the opening surfaces of the channels 3. For such purpose, said one surface 1a of the base member 1, that is a lower surface of the cover plate 2 which surface is joined to the joint surface preferably has a flatly and smoothly finished surface. In the state where the cover plate 2 has been bonded to the base plate 2, a plurality of through holes 4 are provided to the cover plate 2 so that the through holes correspond to each of the channels 3 in order that the fluid (for example, an analyte) is transferred into and from each of the channels 3. For example, four through holes 4 are provided in the form of circular bores as shown in the drawings, each through hole corresponds to each of ends (the right and left ends) of each channel 3.

The base member 1 and the cover plate 2 are bonded together though one piece of the double-faced adhesive sheet 6 between the joint surfaces of them (1 and 2). As shown in FIG. 3, the double-faced adhesive sheet 6 is formed of a sheet substrate 7 and adhesive layers (8 and 8) which are laminated together onto both of the surfaces of the sheet substrate respectively. The double-faced adhesive sheet 6 has two openings 9 through it which correspond to a pattern of the opening surfaces of the channels 3 of the base member 1. Such pattern preferably has two through openings 9 which are of the same dimensions as well as the same shapes as those of the opening surfaces of the channels 3 of the base member 1.

In the usual double-faced adhesive sheet, paper or a non-woven fabric is used as the sheet substrate, of which surface is not smooth and which has not sufficient elasticity (i.e. no self-shape retaining property). Using such double-faced adhesive sheet to bond the base member 1 and the cover plate 2 together, a region where air remains tends to be formed between the base member 1 or the cover plate 2 and the adhesive sheet 6, so that completely sealing of the channels 3 may not be possible. In addition, the region which contains air shows apparent coloration differently from a completely bonded region, so that appearance of the fluid vessel becomes worse. Further, since the sheet substrate has less elasticity and it tends to bend, there is a problem in that locating of the opening 9 of the double-faced adhesive sheet with respect to the channels 3 of the base member 1 is very difficult upon the base member 1 and the cover plate 2 are joined.

In order to eliminate the above mentioned insufficient joint and non-readily handling induced by the sheet substrate, it is preferable in Example 1 to use, as a sheet substrate 7, a flat and smooth plastic sheet which has an elasticity so that it does not easily bend and further which has a chemical resistance. For example, using a sheet material made of a polyethylene terephtalate, the sheet substrate 7 is prepared. The thickness of the sheet substrate 7 depends on the material of which it is made, and it may be in the range between about 0.02 mm and about 0.05 mm, and the thickness of each adhesive layer 8 may be 0.05 mm. The double-faced adhesive sheet 6 has a projected planar shape which is generally the same as that of the base member 1 so that the base member 1 and the cover plate 2 are bonded together through the whole of their joint surfaces thereof except the areas where the channels 3 are located. It is noted that the sheet substrate may be made of a metal sheet, for example, an aluminum sheet, a stainless sheet or a glass sheet.

When the cover plate is bonded to the base member 1, a first connecting tool is prepared which includes two holding members which can be inserted into the channels 3, respectively. Each holding member is inserted into each of the openings 9, whereby the double-faced adhesive sheet 6 is load onto the first connecting tool. Then, ends of the two holding members are fixed into the channels 3 of the base member 1, and the double-faced adhesive sheet 6 is located onto the base member 1, followed by pressing a whole of the lower surface the double-faced adhesive sheet 6 against the base member 1 so as to bond them together. Thereafter, the first connecting tool is removed from the base member 1.

Then, using a second bonding tool, the cover plate 2 is located with respect to the base member 1, followed by pressing the joint surface (i.e. a lower surface) of the cover plate 2 against an upper surface of the double-faced adhesive sheet 6 so as to bond them together. Thereafter, the second bonding tool is removed from the base member 1, and thereby the fluid vessel is completed. Since the base member 1 has its projected planar shape which is the same as that of the cover plate 2, the base member 1 and the cover plate 2 are appropriately located as to each other by bringing their peripheries together while being aligned.

With the completed fluid vessel, the fluid (such as an analyte) can be supplied into each channel 3 via the through hole 4 of the cover plate 2, and then the through hole 4 may optionally be closed. The fluid in the channel may be discharged via the through hole 4.

Upon using the fluid vessel, no unwanted space is formed around the opening of the each channel 3 since the space between the cover plate 2 and the periphery of the opening of each channel 3 is occupied by the inner peripheral wall portion of the opening 9 of the both-faced adhesive sheet 6. Portions of the adhesive layers 8 and 8 which portions face to the channel 3 are limited to only exposed surfaces of the inner peripheral wall portions, so that a chance of the adhesive layers 8 and 8 to be in contact with for example the analyte can be minimized. Therefore, clouding of the analyte and also reduction of analysis accuracy caused by a chemical reaction both caused by dissolution of the adhesive layers 8 into the analyte can be suppressed as much as possible.

When the inner peripheral wall portion of the opening 9 of the double-faced adhesive sheet 6 is coated with a resin which is chemically resistive, the dissolution of the adhesive layers 8 and 8 and the unwanted reaction of the adhesive layers 8 with the analyte are more surely avoided.

### EXAMPLE 2

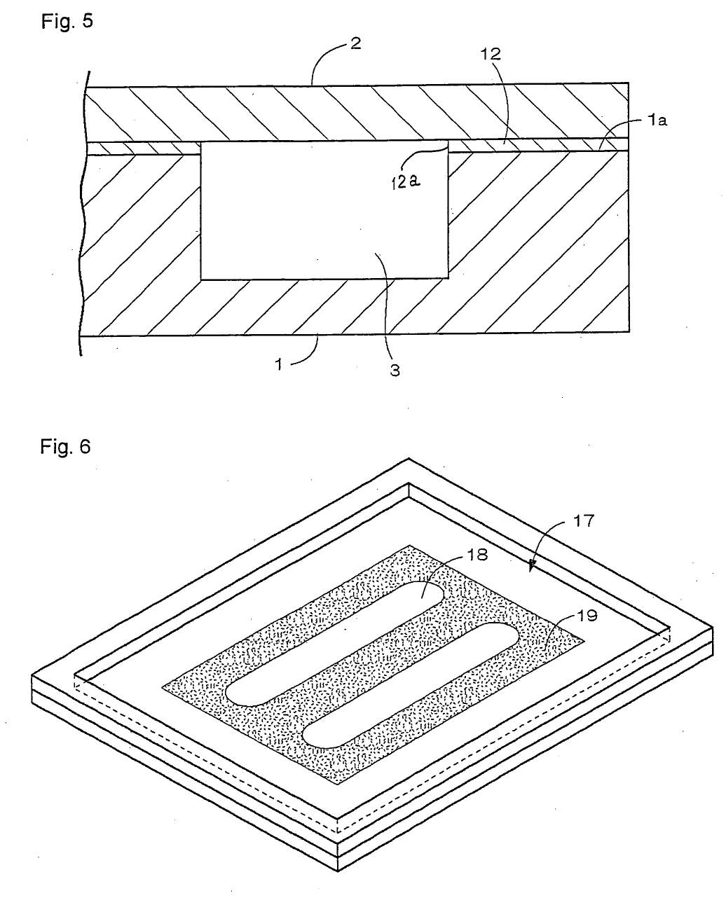

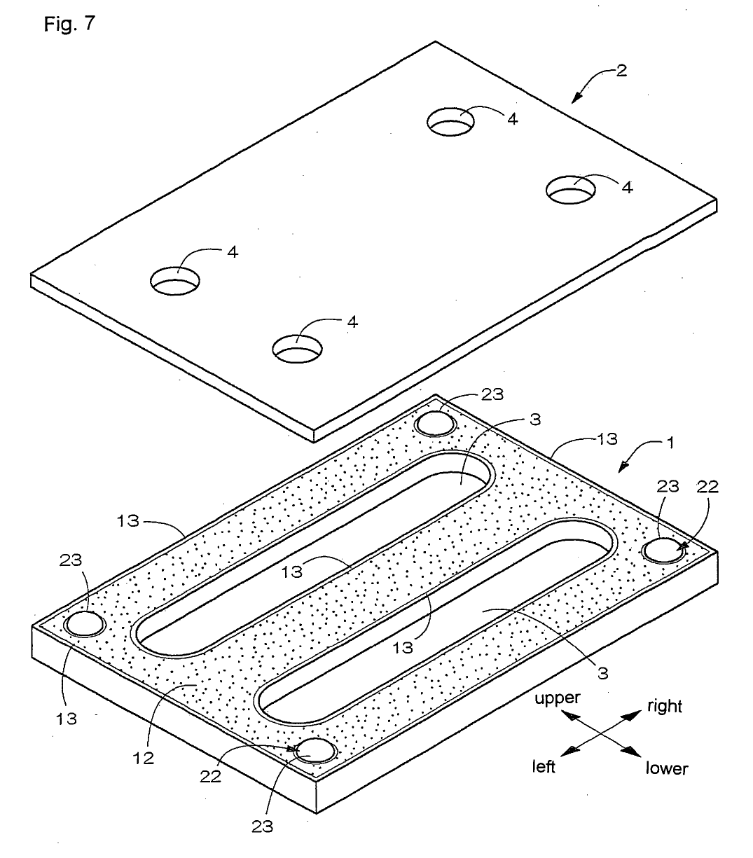

With reference to FIGS. 4 to 6, Example 2 of the fluid vessel (for example, a reaction vessel) according to the present invention which corresponds to the second aspect will be explained. It is noted that FIG. 4 schematically shows a perspective view of the fluid vessel in its exploded state, FIG. 5 schematically shows a plan view of a portion of the fluid vessel, and FIG. 6 schematically shows a perspective view of a stencil plate which is used for the formation of an adhesive layer. In Example 2, since the base member 1 and the cover plate 2 which is boned to the base member 1 are substantially the same as those in Example 1, the same reference numbers as in Example 1 are used to indicate the same members as in Example 1, and explanations of them are omitted here. It is noted that a whole of the cover plate 2 is made of a transparent material for the purpose of passing UV as explained below.

In FIGS. 4 and 5, upon the production of the fluid vessel, the base member 1 and the cover plate 2 are bonded together through an adhesive layer 12. The adhesive layer 12 is made of for example an acrylic based UV curable adhesive or an epoxy based UV curable adhesive, and is formed on the joint surface (said one surface 1a) of the base member 1 by application of the screen printing manner. Since the UV curable adhesive does not initiate its curing without being irradiated with UV, location of the base member 1 with respect to the cover plate 2 may be appropriately adjusted even after the cover plate 2 is placed on the base member 1.

As shown in FIG. 6, the stencil plate 17 which is used for the screen printing manner has non-printing regions 18 of which formation pattern is the same as the formation pattern of the opening surfaces of the channels 3 as well as a printing region 19 which surrounds the non-printing regions 18, and those regions are formed by, for example, the photoengraving manner. Such stencil plate allows an amount of the adhesive to pass through only the printing region 19 so as to be applied to the joint surface of the base member 1.

When the adhesive layer 12 is formed on the base member 1 in the screen printing manner, the adhesive can be accurately printed on the joint surface without deviation except the areas corresponding to the opening surfaces of the channels 3, and also the thickness of the adhesive layer 12 can be set to be uniform. The thickness of the adhesive layer 12 may be arbitrarily controlled by appropriately selecting a mesh size of a screen for the stencil plate 17, a tension of the screen, a printing pressure, a squeegee type, and the like. Considering the dissolution of the adhesive into the fluid, the thickness is preferably as small as possible. In Example 2, the thickness of the adhesive layer 12 is set to be for example 40 μm.

After forming the adhesive layer 12 on the joint surface of the base member 1, the cover plate 2 is located as to the base member 1 with using a connecting tool, and the joint surfaces of these 1 and 2 are joined with each other through the adhesive layer 12. Upon such joining, when the base member 1 has its projected planar shape which is the same as that of the cover plate 2, they are appropriately located to each other by bringing their outer peripheries together while aligning them.

The irradiation of UV is carried out from the above of the cover plate 2 for a predetermined period while keeping the above mentioned joined state, whereby curing the whole of the adhesive layer 12, so that the base member 1 and the cover plate 2 are integrated together, which results in the completed fluid vessel. Since the cover plate 2 is made of the transparent material, the whole of the adhesive layer 12 can be irradiated with UV effectively. In addition, since a curing state of the adhesive, or a reaction state of the analyte in the channel 3 can be observed through the transparent cover plate 2, for example prevention of insufficient curing of the adhesive or stable analysis can be possible.

With thus produced fluid vessel, since only the peripheral portion (precisely, an inner peripheral side surface 12a) of the opening of the adhesive layer 12 is exposed to the channel 3 as shown in FIG. 5, the contact chance of the adhesive layer 12 with the fluid such as an agent (for example, a liquid for analysis), an analyte, or the like becomes minimized, so that for example, the clouding of the liquid for analysis and also the reduction of analysis accuracy caused by the chemical reaction both induced by the dissolution of the adhesive can be suppressed as much as possible. When the adhesive layer 12 is made of an epoxy based UV curable adhesive which is excellent in its chemical resistance, for example such clouding of the liquid for analysis can be prevented more surely. The peripheral portion of the opening of the adhesive layer 12 is located at almost the same level as that of the opening surface of the channel 3, no unwanted space is formed around the opening surface of the channel 3.

It is noted that the adhesive layer 12 is preferably formed with the UV curable adhesive, but it is possible to form the adhesive layer 12 by printing for example an thermosetting adhesive which contains no solvent.

### EXAMPLE 3

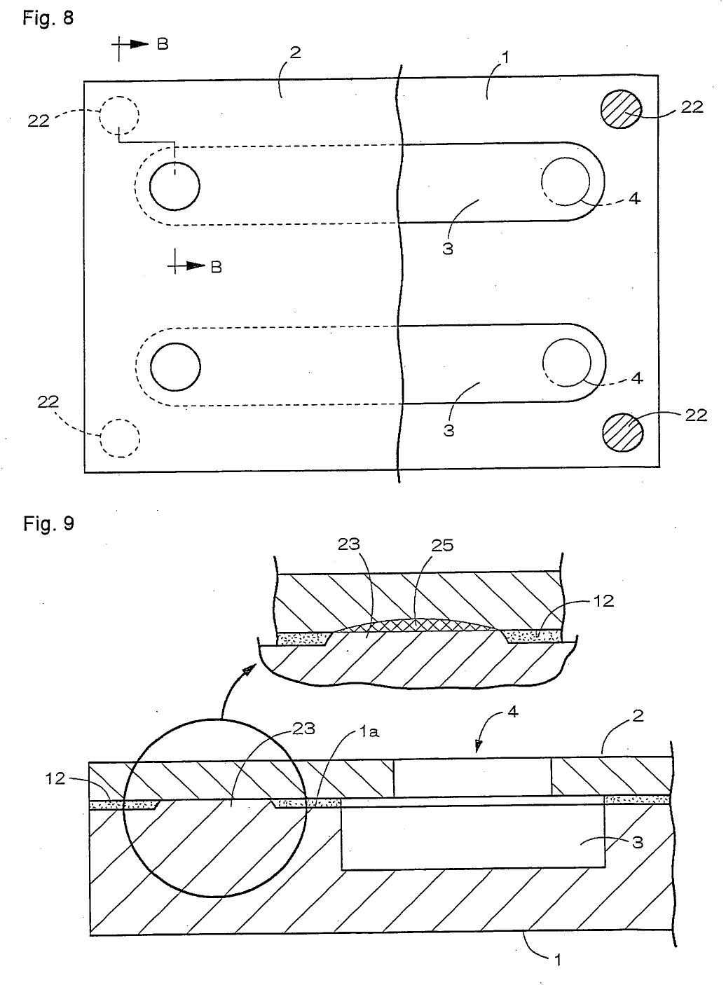

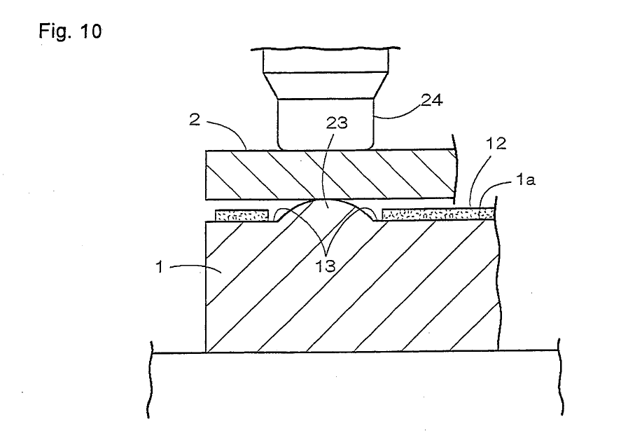

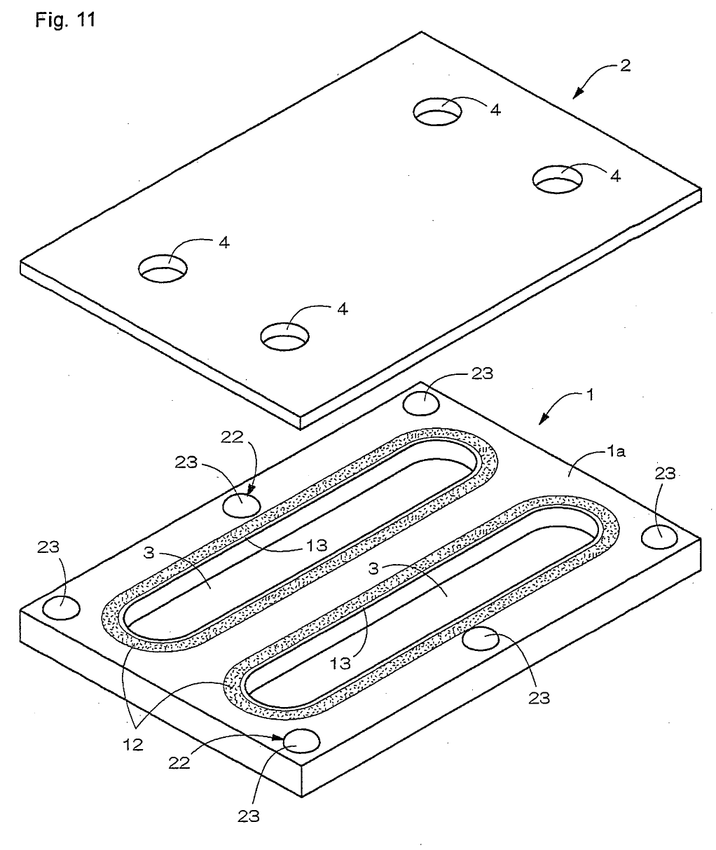

With reference to FIGS. 7 to 10, Example 3 of the fluid vessel (for example, a reaction vessel) according to the present invention which corresponds to the second aspect will be explained. It is noted that FIG. 7 schematically shows a perspective view of the fluid vessel in its exploded state, FIG. 8 schematically shows a plan view of the fluid vessel while a part thereof is cut away, and FIG. 9 schematically shows a cross sectional view of a portion of the fluid vessel (along the line B-B in FIG. 8), and FIG. 10 schematically shows in a cross-sectional view how to carry out spot-welding. In Example 3, since the base member 1 and the cover plate 2 which is boned to the base member 1 are substantially the same as those in Examples 1 and 2, the same reference numbers as in the Examples are used to indicate the same members as in the Examples, and explanations of them are omitted here. It is noted that each of the base member 1 and the cover plate 2 is a molded article of a thermoplastic material.

In FIG. 7, an adhesive layer 12 is formed on one surface 1a of the base member 1 by applying an adhesive onto generally whole of said one surface. In the state where the cover plate 2 is placed on the base member 1, the space between the opposite surfaces (i.e. the joint surfaces) of the base member 1 and the cover plate 2 is filled with the adhesive layer 12 so that sealing between them is ensured. Upon applying the adhesive, relief margins 13 are provided along the outer periphery of the joint surface (said one surface 1a) of the base member 1 and also along the peripheries of the openings of the channels 3 respectively so that the adhesive can spread over the relief margins when the cover plate 2 is placed on the base member 1. The relief margins 13 correspond to regions of the base member 1 where no adhesive is applied when the application of the adhesive layer 12 is carried out onto the base member 1. In Example 3, the adhesive layer 12 is formed with for example a UV curable adhesive.

A spot-welded portion 22 is provided around each of the four corners on one surface 1a of the base member 1 on which portion no adhesive is applied. At the spot-welded portions, the base member 1 is spot-welded with the cover plate 2 to connect them together. On the base member 1, a small concave portion 23 is provided on each of the spot-welded portions 22 on the base member 1 as shown in FIGS. 7 to 10. It is preferable that a relief margin 13 for the adhesive layer 12 is also provided around each of the small convex portions 23.

Referring to FIG. 10, when the cover plate 2 is joined with the base member 1, the adhesive layer 12 is formed on one surface 1a of the base member 1 by the application thereof as in Example 1, and then the cover plate 2 is placed on the base member 2 so that the tops of the small convex portions 23 allows to be in contact with the joint surface of the cover plate 2. In thus state, four horns 24 of an ultrasonic welding machine press the small convex portions 23 respectively through the cover plate 2 simultaneously, and a portion of the top of each of the convex portions 23 is melted or softened by means of vibration/friction heat between the base member 1 and the cover plate 2. During such spot-welding, since the small convex portions 23 are surrounded by the adhesive layer 12, there is no problem in that dusts (melt particles) generated by the ultrasonic melting enter the channels 3.

In FIG. 9, reference number 25 indicates the top of the small convex portion 23. The top 25 of the small convex portion 23 which is in the softened or molten state is fused with the joint surface of the cover plate 2, and it is solidifies to be in the state in which the joint surfaces of the base member 1 and the cover plate 2 are linked to each other.

By means of the above spot-welding, the cover plate 2 is securely caught by the base member 1 at the spot-welded portions 22. Since the tops 25 of the small convex portions 23 melt and spread to fill the gaps between the base member 1 and the cover plate 2, the cover plate 2 is firmly caught by the base plate through the solidification of the resin.

A pressing force of the horns 24 against the cover plate 2 is set such that the whole of the joint surface of the cover plate 2 is sufficiently in contact with the whole of the adhesive layer 12. When pressing with the horns, the adhesive layer 12 is pressed, and it is extended and spread a little between the base member 1 and the cover plate 2. As shown in FIGS. 7 and 10, since the relief margins 13 for the adhesive layer 12 are provided between the joint surfaces of the base member 1 and the cover plate 2, no adhesive layer 12 penetrates into the channels 3 or protrudes out from the fluid vessel.

Thereafter, the adhesive layer 12 is irradiated with UV through the transparent cover plate 2 so as to cure the adhesive layer 12, whereby the joint surfaces of the base member 1 and the cover plate 2 are bonded together. When the cover plate 2 is made of the transparent material, the whole of the adhesive layer 12 is effectively irradiated with UV. In addition, an curing extent of the adhesive may be checked through the cover plate 2, or a reaction state of an analyte in the channel 3 can be observed through the transparent cover plate 2, and therefore insufficient curing of the adhesive is prevented and also stable analysis becomes possible. Thus curing of the adhesive layer 12 sufficiently fills the space between the joint surfaces of the base member 1 and the cover plate 2, so that better sealing between them can be ensured.

Since, in advance of the curing of the adhesive layer 12, the base member 1 is integrated with the cover plate 2 at the four points around the corners by the spot-welding, no holding of the base member 1 and the cover plate 2 for example with a tool is required to be kept upon the curing operation of the adhesive layer 12, so that the productivity of the fluid vessel is improved correspondingly, which reduces the production cost of the fluid vessel. Locating and holding the base member 1 and the cover plate 2 is not required until curing of the adhesive layer 12 is completed, which reduces the necessary man-hours so as to contribute to the reduction of the production cost of the fluid vessel. In addition, the combination of the face to face bonding by means of the adhesive layer 12 with the point-to-point bonding by means of the spot-welding can securely integrate the base member 1 and the cover plate 2 together, so that the base member 1 and the cover plate 2 will not be separated even with an application of a great dropping impact force.

### EXAMPLE 4

With reference to FIG. 11, Example 4 according to the present invention in which Example 3 is partly modified will be explained. In Example 4, the adhesive layers 12 and the relief margins 13 are partly provided only around the peripheries of the above mentioned channels 3 on said one surface 1a of the base member 1 so that the channels are rimmed by the adhesive layers 12 and the relief margins 13 respectively, which is different from above explained Example 3. In addition to the spot-welded portions around the four corners, two spot-welded portions 22 each having a small convex portion 23 are also provided at intermediate positions along a length direction of the base member 1, which is also different from above explained Example 3. The other features are the same as those in Example 3, and therefore the same reference numbers as in Example 3 are used to indicate the same members as in Example 3, and explanations of them are omitted here.

According to Example 4, the fluid vessel which is excellent in its sealing property is produced less expensively as in Example 3. Since an amount of the adhesive to be applied which is expensive is reduced, the fluid vessel can be provided with a correspondingly less expensive cost.

In Examples 3 and 4, the number as well as the arrangement of the small convex portions 23 may be optionally changed when necessary. Further, the small convex portions 23 may be provided to not the base member 1, but to the cover plate 2. In any one of the provision of the adhesive layer 12 to the base member 1 and the provision of the adhesive layer 12 to the cover plate 2, it is required to provide the adhesive layer 12 except the small convex portions 23. In Examples 3 and 4, the spot-welding is carried out with the ultrasonic welding, but it may be carried out with the heat welding (or heat fusion).

In Examples 1 to 4, the size, the shape, the number, and the pattern of the channel(s) 3 which is to be formed in the base member 1 may be optionally changed as required. In connection with this, the channels 3 may be communicated with each other by for example forming a small channel(s) between them.

1. A fluid vessel comprising:
a base member having a channel on its one surface which channel is able to receive a fluid;
a cover plate which closes an opening surface of the channel and which is located above the base member; and
a bonding element which joins the base member and the cover plate,
wherein the bonding element is a double-faced adhesive sheet which is placed between a joint surface of the base member and a joint surface of the cover plate,
the double-faced adhesive sheet has an opening which corresponds to the opening surface of the channel of the base member, and
a space between the cover plate and a periphery portion of the channel which portion defines the opening surface of the channel is occupied by an inner peripheral wall portion which defines the opening of the double-faced adhesive sheet.

2. The fluid vessel according to claim 1 wherein the double-faced adhesive sheet is composed of a sheet substrate made of a plastic sheet and adhesive layers each of which is laminated integrally onto each of surfaces of the sheet substrate.

3. The fluid vessel according to claim 1 wherein a surface of said inner peripheral wall portion of the double-faced adhesive sheet has a resin coating which is chemically resistive.

4. The fluid vessel according to claim 1 wherein the base member is in the form of a rectangular or square plate, and the cover plate has a projected planar shape which is the same as that of the base member.

5. The fluid vessel according to claim 1 wherein the joint surface of the base member and the joint surface of the cover plate are flat and smooth respectively.

6. The fluid vessel according to claim 1 wherein the fluid is a liquid.

7. A fluid vessel comprising:
a base member having a channel on its one surface which channel is able to receive a fluid;
a cover plate which closes an opening surface of the channel and which is located above the base member; and
a bonding element which joins the base member and the cover plate,
wherein the bonding element is an adhesive layer which is formed on at least one of a joint surface of the base member and a joint surface of the cover plate, and
the adhesive layer is formed on at least one of the joint surfaces except the opening surface of the channel in a printing manner.

8. The fluid vessel according to claim 7 wherein the cover plate is made of a transparent material, and an adhesive of the adhesive layer is an ultraviolet curable adhesive.

9. The fluid vessel according to claim 8 wherein the adhesive of the adhesive layer is an epoxy based ultraviolet curable adhesive which is chemically resistive.

10. The fluid vessel according to claim 7 wherein the adhesive layer is formed on the joint surface of the base member.

11. The fluid vessel according to claim 7 wherein the base member is in the form of a rectangular or square plate, and the cover plate has a projected planar shape which is the same as that of the base member.

12. The fluid vessel according to claim 7 wherein the joint surface of the base member and the joint surface of the cover plate are flat and smooth respectively.

13. The fluid vessel according to claim 7 wherein the fluid is a liquid.

14. A fluid vessel comprising:
a base member having a channel on its one surface which channel is able to receive a fluid;
a cover plate which closes an opening surface of the channel and which is located above the base member; and
a bonding element which joins the base member and the cover plate,
wherein the bonding element is an adhesive layer which is formed on at least one of a joint surface of the base member and a joint surface of the cover plate, and
each of the base member and the cover plate is made of a thermoplastic material, and the base member and the cover plate are spot-welded at least at two points.

15. The fluid vessel according to claim 14 wherein the joint surface of the base member and the joint surface of the cover plate are flat and smooth respectively except regions where spot-welded is performed.

16. The fluid vessel according to claim 14 wherein one of the joint surface of the base member and the joint surface of the cover plate comprises a spot-welded portion which has a convex portion which abuts against the other of the joint surface of the base member and the joint surface of the cover plate.

17. The fluid vessel according to claim 14 wherein the cover plate is made of a transparent material, an adhesive of the adhesive layer is an ultraviolet curable adhesive, and the adhesive layer is cured by UV irradiation after the base member and the cover plate are spot-welded at the spot-welded points.

18. The fluid vessel according to claim 14 wherein one surface of the base member comprises the adhesive layer and a spot-welded portion which includes a convex portion, and the adhesive layer is formed around a periphery of the channel except the spot-welded portion.

19. The fluid vessel according to claim 14 wherein a spot-welded region is spot-welded by an ultrasonic welding manner.

20. The fluid vessel according to claim 14 wherein the base member is in the form of a rectangular or square plate, and the cover plate has a projected planar shape which is the same as that of the base member.

21. The fluid vessel according to claim 14 wherein the fluid is a liquid.

22. The fluid vessel according to claim 1 which is a reaction vessel.

23. The fluid vessel according to claim 7 which is a reaction vessel.

24. The fluid vessel according to claim 14 which is a reaction vessel.
